(19) United States
(12) Patent Application Publication
(10) Pub. No.: US 2022/0265988 A1
(43) Pub. Date: Aug. 25, 2022

(54) DRAINAGE CATHETER WITH SAFETY VALVE

(71) Applicant: NORCRI INVEST, S.L., Las Palmas de Gran Canaria (ES)

(72) Inventor: Norberto Santana Rodríguez, Las Palmas de Gran Canaria (ES)

(21) Appl. No.: 17/622,100

(22) PCT Filed: Jun. 24, 2020
(86) PCT No.: PCT/EP2020/067739

§ 371 Date: Dec. 22, 2021

(30) Foreign Application Priority Data
Jun. 25, 2019 (EP) 19382534.6

(51) Int. Cl. A61M 39/22; A61M 27/00

(52) U.S. Cl. CPC A61M 39/227; A61M 27/00; A61M 25/0108

(57) ABSTRACT

The disclosure relates to the area of medicine and surgery, in particular it pertains to a drainage catheter (100) that comprises a high capacity drain comprising a radiological guide (110a) with a suction mesh (110b), a safety control opening/closing system (160), an internal pneumatic clamping system (140) and/or an external adhesive skin protection system (150). A reservoir for the administration of local anesthetics and antibiotics may be additionally provided. The disclosed catheter system optimizes flow surface, facilitates procedures for handling and removal of drainage, reduces the risk of occlusions, pain, injuries and infections of the surrounding skin and maintains sufficient structural strength to prevent collapses, among others.

### BACKGROUND

### 1. Field of the Disclosure

The disclosure relates to drainage catheters and more specifically to a drainage catheter with a safety valve.

### 2. Background Information

Medical drainage catheters have been used for over 40 years to drain fluid or air from various body cavities. Some applications for medical drainage catheters include placement within the thorax for drainage of gas to treat pneumothorax, placement within the thorax to drain fluid, placement within abscesses to drain purulent fluid, placement within the urinary tract to drain urine or purulent fluid in cases of urinary tract obstruction, and placement within the biliary tree to drain bile or purulent fluid in cases of biliary obstruction.

Current drainage catheters do not have safety control systems for opening/closing them which requires a degree of external manipulation for clamping or for the administration of treatments. For example, drainage catheters must be manipulated and/or clamped to skin for the administration of treatments by using implements including but not limited to tweezers, plugs and/or stitches.

Certain risks may be associated with treatment using such implements. For example, surgical drains require fixation to the skin with stitches, which causes pain and discomfort at the insertion site (both internally and externally) and promotes infection of the surrounding tissues or within the cavities.

As such, a need exists in the art for medical drainage catheters that overcome or ameliorate one or more of the disadvantages known in the art.

### BRIEF DESCRIPTION OF THE FIGURES

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIG. 1A-1B illustrates a catheter according to a disclosed embodiment;

FIGS. 2A-2E illustrate a valve for a catheter according to a disclosed embodiment; and

FIGS. 3A-3B illustrate the valve attached to the catheter with the valve in opened and closed states according to a disclosed embodiment.

### DETAILED DESCRIPTION

The disclosure relates to the area of medicine and surgery, pertains to the field of a drainage catheter that comprises a high capacity drain with a 360 degree multi-perforated suction mesh, a safety control opening/closing system, an internal pneumatic clamping system, and an external adhesive skin protection system with or without reservoir for the administration of local anesthetics and antibiotics to be used in the fields of medicine and veterinary. The disclosed catheter system optimizes flow surface, facilitates procedures for handling and removal of drainage, reduces the risk of occlusions, pain, injuries and infections of the surrounding skin and maintains sufficient structural strength to prevent collapses, among others.

The present disclosure now will be described more fully hereinafter, but not all embodiments of the disclosure are shown. While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular structure or material to the teachings of the disclosure without departing from the essential scope thereof.

The drawings accompanying the application are for illustrative purposes only. They are not intended to limit the embodiments of the present application. Additionally, the drawings are not drawn to scale. Elements common between figures may retain the same numerical designation.

Where a range of values is provided, it is understood that each intervening value between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of’ and “consisting essentially of’ shall be closed or semi-closed transitional phrases, respectively, as set forth in the 10 United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. It should also be understood that, unless clearly indicated to the contrary, in any methods claimed herein that include more than one step or act, the order of the steps or acts of the method is not necessarily limited to the order in which the steps or acts of the method are recited.

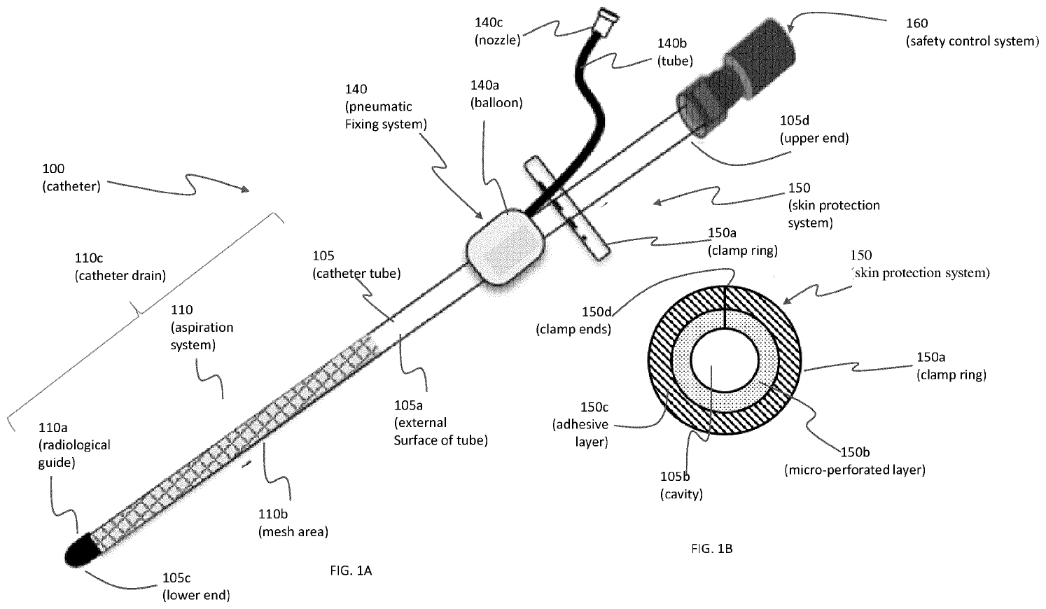

Turning to FIGS. 1A and 1B, the disclosure relates to a catheter 100 which is a drainage catheter. The catheter 100 may be straight or curved, flexible or rigid, and may have different diameters and lengths. The catheter 100 may comprise a catheter tube 105 with an external surface 105a and an internal cavity 105b, an axial lower end 105c and an axil upper end 105d.

The catheter 100 includes a first system 110 which is a high capacity aspiration system. The aspiration system 110 includes radiological guide 110a and a 360 degrees (annular cross section, circumferentially enclosing the tube 105) mesh suction area 110b (also referred to as a suction mesh 110) extending axially along the tube 105. The mesh 110b has a small pore size that allows drainage of the cavity 105b, increasing suction capacity and minimizing the risk of occlusion.

In certain embodiments, the mesh 110b comprises relatively small pores, for example, pores of about 0.05-5 mm, which extend axially along the first four to eight internal centimeters of the axial lower end 105c of the tube 105 to define a catheter drain 110c. This allows enhancing suction capacity and reducing the risk of occlusion due to the aspiration of solid materials such as blood, fibrin or detritus from the cavity 105b where the drain is accommodated, while allowing a greater intra-cavitary diffusion of the therapeutics liquids applied through the drain 110c. The drain 110c is composed by silicone, polyurethane or any other rigid or flexible material. The mesh area 110b is additionally composed of an opaque (radiopaque) material that facilitates identification and positioning of the drain 110C.

In certain embodiments, the catheter 100 includes a second system 140 which is an internal pneumatic clamping system (also referred to a pneumatic fixing system). The clamping system 140 is disposed on the external surface 105a of the tube 105. The clamping system 140 includes an inflatable balloon 140a connected to a tube 140b with an air inlet nozzle 140c. The clamping system 140 allows attachment to surrounding tissues and plugging a wound without points of attachment to skin.

More specifically, the balloon 140a, allows attachment to the surrounding tissues under the skin and plugging the wound without points of attachment to the skin. This balloon 140a may be non-perforated and externally connected to an inflation key, that is, an inlet nozzle 140c, that allows its expansion under the skin, immobilizing the drain 110c and preventing air entry or exit of air and fluid from the cavity 105b. The balloon 140a may alternatively be micro-perforated, having a micro-perforated area (not illustrated) that is exterior to the cavity 105b and allows diffusion of antibiotics, local anesthetics or any other element into the deep layers of the surgical wound.

In additional embodiments, the catheter 100 includes a third system 150 which is a skin protection system. The skin protection system 150 is an external adhesive, hypoallergenic, mobile, disposable clamping annulus or ring 150a that is adaptable to provide skin protection with or without an internal reservoir. The ring 150a is formed from a micro-perforated sub-layer 150b in a central area and an adhesive layer 150c for providing passive diffusion of antibiotics or local anesthetics to the wound area and surrounding tissues. The ring 150a is capable of being clamped about circumferential ends generally referred to as 150d. The ring 150a may also be replaceable.

The ring 150a is movable and longitudinally adaptable, axially along the tube 105, to provide skin protection. The ring 105a provides external fixation, with or without an internal reservoir that surrounds the catheter tube 105. The ring 105a allows a higher adaptability and an increasing clamping of the catheter 100 and protection to skin surrounding the wound in which the catheter 100 is introduced.

The clamp 150a may be connected to a stopcock through which antibiotics, local anesthetics or other therapeutic substances may be delivered into the surgical wound where it is fixed. In addition, the clamp 150a may be closed by stitches at the clamp ends 150d to increase fixation, if desired.

In certain embodiments, the catheter 100 includes a fourth system 160 which is a safety control system (also referred to as a safety valve 160) for controlling opening and closing of the catheter. The safety valve 160 allows for administration of treatments in a simple, fast, safe, and efficient way.

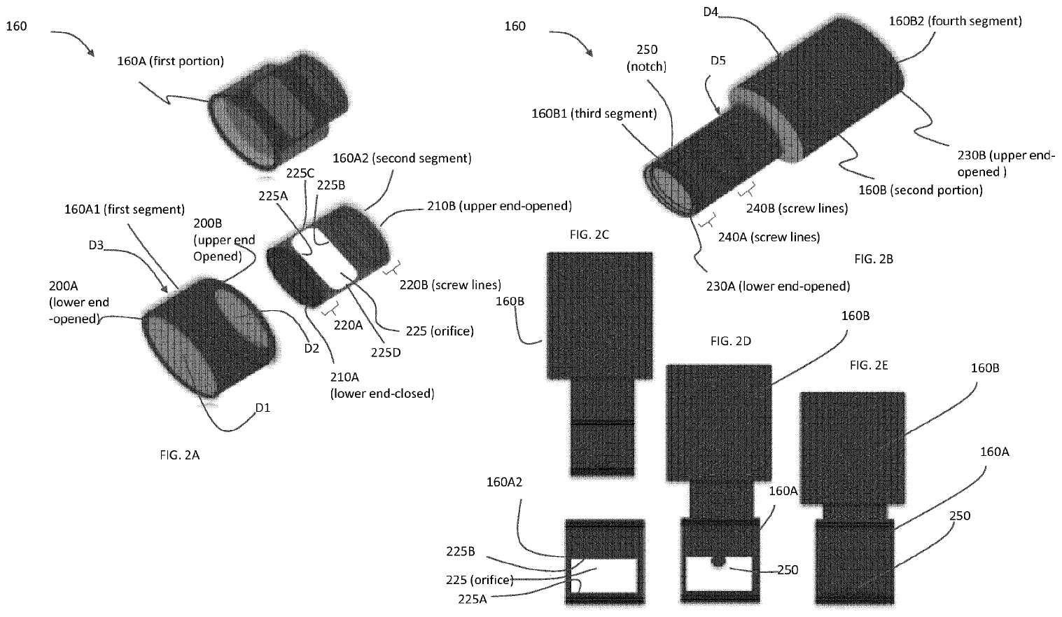

More specifically, turning to FIGS. 2A-2C, the safety valve 160 forms a safety control valve that includes a pair of valve portions including a first portion 160A and a second portion 160B respectively defining an axially lower portion an axially upper portion. Turning first to the first portion 160A, there are two segments that are cylindrical and include a first segment 160A1 and a second segment 160A2 that respectively define an axially lower segment and an axially upper segment.

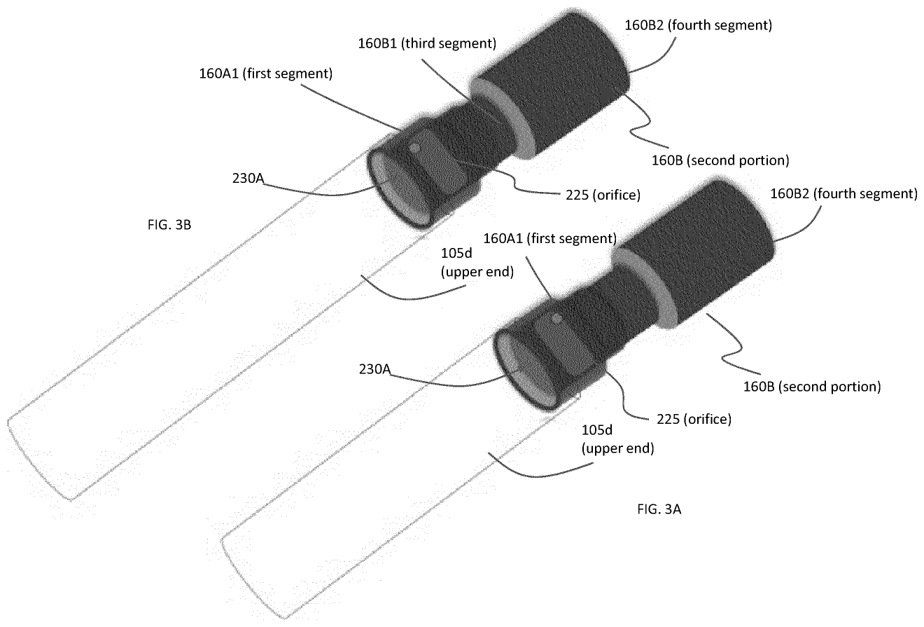

The first segment 160A1 is open on both axial ends generally referred to as 200, including a first end 200A and a second end 200B that respectively define an axially lower end and an axial upper end. The axially lower end 200A has a first diameter D1 sized to fit over or within the upper end 105d of the catheter tube 105 (see FIGS. 3A and 3B). The axially upper end 200B has a second internal diameter D2 that is smaller than the first diameter D1 and is sized to receive the second segment 160A2. An outer diameter D3 for the first segment 160A1 may be constant.

The second segment 160A2 has opposing axial ends generally referred to as 210 including a first end 210A and a second end 210B respectively defining an axially lower end 210A and an axially upper end 210B. The axially upper end 210B is open and the axially lower end 210A is closed. The closed lower end 210A limits a lower displacement of the second portion 160B into the second segment 160A2. Two sets of internal screw threads generally referenced as 220 including a first set of screw threads 220A and a second set of screw threads 220B are provided at the respective opposing lower and upper ends 210A, 210B. In addition, a circumferential orifice 225 (discussed in greater detail below) is provided that is intermediate the sets of screw lines 210. The orifice 225 includes an upper edge 225A and a lower edge 225B to define an axial span of the orifice 225, and a first circumferential end 225C and a second circumferential end 225D to define an annular span of the orifice 225.

The second portion 160B includes a cylinder including two segments, including a third segment 160B1 and a fourth segment 160B2 respectively defining an axially lower segment and an axially upper segment. The fourth segment 160B2 has a fourth diameter D4 that is an outer diameter. The fourth diameter D4 is greater than a fifth diameter D5, which is an outer diameter for the third segment 160B1.

The second portion 160B is open at both ends, generally referred to as 230 including a first end 230A and a second end 230B defining a lower end and an upper end. The third segment 160B1 includes two sets of external screw threads generally referenced as 240 including a third set of screw threads 240A and a fourth set of screw threads 240B. The screw threads 240 are externally provided proximate the first end 230A intermediate an axial length of the third segment 160B1. The screw threads 240 mate in the second portion 160B mate with the screw threads 220 in the first portion 160A for interconnecting the portions to form the safety control valve 160.

Turning to FIGS. 2C-2E, the third segment 160B1 includes an outwardly facing notch 250 or protrusion prevent separation of the first portion 160A and the second portion 160B after the two are interconnected. That is, the notch 250 protrudes into the orifice 250 in the first portion 160A to provide an axial lower limit (FIG. 2D) and an upper limit (FIG. 2E) at the respective axial lower and upper edges 225A and 225B of the orifice 250. At the axial upper limit, when the notch 250 is against the axial upper edge 225B of the orifice 225, the second screw threads 220B engage the third screw threads 240A. At the lower limit, when the notch 250 is against the axial lower edge 225A of the orifice 225, the first screw threads 220A engage the third screw threads 240A and the second screw threads 220B engage the fourth screw threads 240B. In addition, as illustrated in FIG. 2D, at the upper limit, the orifice 250 is opened, and the safety valve 160 is in an open state. As illustrated in FIG. 2E, in the lower limit, the orifice 250 is closed and the safety valve 160 is in a closed state.

The annular span of the orifice 225 is such that a securing grip may be obtained between the sets of threads 220 and 250 when the first portion 160A and the second portion 160B are being interconnected. For example, the annular span of the orifice 225 may be twenty five to fifty percent of the circumference of the second upper 160A2. The operation of the safety valve 160 is based on the displacement of the second portion 160B relative to the first portion 160A to the open and closed states previously described, so that the turns of the thread threads provide and maintain the open and closed states.

With the safety valve 160 is in the opened state (FIGS. 2D and 3A), the fluid or air (both referred to as “fluid”) will pass flow in an axially upward direction from the tube 105, through the first segment 160A1 and out of the orifice 225. The closed lower end 230A of the second portion 160B prevents further flow in an axial upward direction. When the safety valve 160 is in the closed state (FIGS. 2E and 3B) the threaded engagement between the sets of threads 220 and 240 along with the closed lower end 230A of the second portion 160B provides a fluid seal that prevents flow axially to and out of the orifice 225.

Those of skill in the art will appreciate that various example embodiments are shown and described herein, each having certain features in the particular embodiments, but the present disclosure is not thus limited. Rather, the present disclosure can be modified to incorporate any number of variations, alterations, substitutions, combinations, sub-combinations, or equivalent arrangements not heretofore described, but which are commensurate with the scope of the present disclosure. Additionally, while various embodiments of the present disclosure have been described, it is to be understood that aspects of the present disclosure may include only some of the described embodiments. Accordingly, the present disclosure is not to be seen as limited by the foregoing description, but is only limited by the scope of the appended claims.

We claim:

1. A catheter comprising:
a catheter body;
a valve comprising a safety control opening/closing system;
at least one of an internal pneumatic clamping system, an external adhesive skin protection system or both; and
a radiological guide comprising a multi-perforated mesh area circumferentially surrounding the catheter body.

2. The catheter of claim 1, wherein the mesh area contains a radiopaque material.

3. The catheter of claim 2, wherein an inflatable balloon is affixed to an outer surface of the body.

4. The catheter of claim 3, wherein the balloon has a micro-perforated external layer.

5. The catheter of claim 4, wherein a clamping ring with an adhesive layer is affixed to the outer surface of the body.

6. The catheter of claim 5, wherein an internal reservoir having a multi-perforated central layer is connected to a stopcock.

7. The catheter of claim 6, wherein a safety valve is fluidly connected to an upper end of the body.
